(19) United States
(12) Patent Application Publication
(10) Pub. No.: US 2014/0236011 A1
(43) Pub. Date: Aug. 21, 2014

(54) METHODS AND SYSTEMS FOR SIMULTANEOUS INTERVENTIONAL IMAGING AND FUNCTIONAL MEASUREMENTS

(71) Applicant: General Electric Company, Schenectady, NY (US)

(72) Inventors: Ying Fan, Niskayuna, NY (US); Hans-Peter Stoll, Waukesha, WI (US)

(73) Assignee: General Electric Company, Schenectady, NY (US)

(21) Appl. No.: 14/266,173

(22) Filed: Apr. 30, 2014

Related U.S. Application Data
(63) Continuation-in-part of application No. 13/600,746, filed on Aug. 31, 2012.

(51) Int. Cl. A61B 8/12; A61B 8/08; A61B 8/00; A61B 5/107; A61B 8/06; A61B 5/00; A61B 5/0205

(52) U.S. Cl. CPC A61B 8/12; A61B 5/0059; A61B 8/488; A61B 5/02055; A61B 5/1076; A61B 8/06; A61B 8/461
USPC 600/440; 600/407; 600/479; 600/454

(57) ABSTRACT

Methods and systems for performing an interventional procedure are presented. A first set of pulses are delivered using at least one image sensor simultaneously with a second set of pulses delivered using at least one flow sensor disposed in an integrated interventional device towards a target region in a subject. Further, structural information corresponding to the target region at a designated time is determined using imaging signals received in response to the first sets of pulses. Additionally, volumetric information corresponding to the target region at the designated time is determined using signals received in response to the second sets of pulses. Moreover, the structural and volumetric information is processed using a determined model to compute one or more diagnostic parameters corresponding to the target region. A diagnostic assessment of the target region is then provided based on the computed diagnostic parameters.

### CROSS-REFERENCE TO RELATED APPLICATION

This application is a continuation-in-part of U.S. patent application Ser. No. 13/600,746, filed Aug. 31, 2012.

### BACKGROUND

Embodiments of the present specification relate generally to interventional procedures, and more particularly to systems and methods for simultaneously performing interventional imaging and functional measurements.

Interventional techniques are widely used for managing a plurality of life-threatening medical conditions. Particularly, certain interventional techniques entail minimally invasive image-guided procedures that provide a cost-effective alternative to invasive surgery. For example, optical coherence tomography (OCT), coronary computed tomographic angiography (CCTA), and/or intravascular ultrasound (IVUS) imaging may be employed as minimally invasive techniques for diagnosing diseased blood vessels using image-derived information. The image-derived information, in turn, may be used to aid medical practitioners in procedures such as angiography and stent placement to restore or increase blood flow to a desired region. Further, the interventional imaging systems may also be used to determine an existence as well as nature and extent of intravascular obstructions, stenosis, and atherosclerosis plaque build-up at particular locations within the blood vessels.

However, not all interventional imaging systems may be similarly suited to identify and evaluate features of interest such as stenotic lesions in different imaging scenarios. CCTA, for example, is widely used to detect a presence or absence of coronary lesions in a blood vessel non-invasively. However, CCTA may not be an appropriate choice for determining a hemodynamic significance of the detected coronary lesions. Additionally, CCTA may occasionally overestimate a severity of the lesion, thus resulting in unnecessary invasive angiography, unneeded revascularization procedures, higher costs, and/or radiation exposure. Accordingly, CCTA finds limited clinical use during revascularization decisions.

Accordingly, certain interventional procedures employ IVUS imaging for diagnosing and/or treating blocked vessels. Typically, IVUS imaging entails use of a miniaturized ultrasound probe including a catheter having a diameter of about 1 millimeter (mm) that may be inserted into, or proximal, a region of interest (ROI) such as a coronary vessel. Particularly, the IVUS catheter may include an imaging sensor such as a side-looking transducer for generating high-frequency sound waves that reflect off tissue or vessel walls. The reflected sound waves are used to generate cross-sectional images from within the vessel for visualizing structural aspects of the vessel in offline or real-time mode.

In one example, IVUS images may be used to assess a need for revascularizing a diseased blood vessel that includes one or more lesions. It may be noted that revascularization of stenotic lesions that induce ischemia are known to improve a patient's functional status and outcome. Conversely, for stenotic lesions that do not induce ischemia, medical therapy alone may be as effective as revascularization without involving any of the associated risks. Estimation of structural characteristics of a stenotic lesion alone, however, may not provide sufficient information for determining which lesions cause ischemia and warrant revascularization or stenting. Accordingly, functional parameters such as fractional flow reserve (FFR), coronary flow reserve (CFR), or percentage area of stenosis may be estimated for assessing a hemodynamic significance of the stenotic lesion. For example, the functional parameters may be estimated for determining a likelihood of the stenotic lesion impeding oxygen delivery to the cardiac muscles of the patient.

Generally, FFR may be defined as a pressure at a distal end of a stenosis relative to an aortic pressure. Conventionally, the aortic pressure is measured using a blood pressure monitor, whereas FFR catheters are used to measure pressure at distal end of lesion. In common clinical practice, an FFR value of less than 0.75 may be used as indication of a severity of the stenosis that may necessitate stenting. For example, an FFR catheter of about 300 micron in size may be advanced across the stenosis to determine a ratio of distal coronary pressure to aortic pressure. The determined ratio may be subsequently used to estimate the FFR and/or pressure gradient corresponding to the fluid flow in a vascular structure of interest. Similarly, CFR may be used to assess functional parameters of coronary arteries. CFR may be defined as the maximum increase in blood flow through coronary arteries above a normal resting volume. Generally, the CFR may be measured post vasodilation, for example, through positron emission tomography or interventional procedures such as transthoracic Doppler echocardiography or coronary catheterization.

In certain interventional procedures, the functional parameters such the FFR and CFR may be used in conjunction with the structural characteristics, for example, determined using IVUS, or computed tomography (CT) imaging for facilitating revascularization decisions. The images acquired using an IVUS catheter may aid in planning and/or post-procedure evaluation of the revascularization procedure by providing information regarding vessel size, lesion distribution, and/or lesion type. Similarly, the FFR estimation using the FFR catheter may be used to provide a functional assessment during procedure planning and post-procedure evaluation of the interventional procedure.

Conventional interventional procedures, thus, may entail use of two different systems that allow for IVUS imaging and functional or flow estimations. Certain other systems incorporate the IVUS and FFR catheters into a single backend system with integrated processing of the data acquired independently by the different IVUS and FFR catheters. Use of such conventional systems, however, entails use of two different catheters (IVUS and FFR) that undergo multiple insertions and retractions into the ROI for measuring structural and functional parameters. The multiple insertions and retractions may result in deviation from a desired measurement site in the vessel, longer procedure time, and patient discomfort. Additionally, advancing the FFR catheter across the stenosis may impede blood flow, thus leading to erroneous velocity and/or pressure measurements. Furthermore, use of the different IVUS and FFR catheters also adds to equipment and operational costs.

### BRIEF DESCRIPTION

In accordance with aspects of the present disclosure, methods, systems, and non-transitory computer readable media that store instructions executable by one or more processors to perform a method for performing an interventional procedure are presented. A first set of pulses are delivered using at least one image sensor simultaneously with a second set of pulses delivered using at least one flow sensor disposed in an integrated interventional device towards a target region in a subject. Further, structural information corresponding to the target region at a designated time is determined using imaging signals received in response to the first sets of pulses. Additionally, volumetric information corresponding to the target region at the designated time is determined using signals received in response to the second sets of pulses. Moreover, the structural and volumetric information is processed using a determined model to compute one or more diagnostic parameters corresponding to the target region. A diagnostic assessment of the target region is then provided based on the computed diagnostic parameters.

One technical effect of the embodiments of methods and systems disclosed and claimed herein includes simplifying the workflow of an interventional procedure by combining an IVUS and an FFR catheter into a single integrated interventional device to provide simultaneous IVUS imaging and flow velocity measurements. The resulting images and/or flow velocity measurements allow for real-time computation of clinically significant parameters such as fluid velocity, flow volume, percentage of stenosis, CFR, and/or FFR. Another technical effect of the present disclosure includes use of a computational fluid dynamics (CFD) model to allow for more accurate lesion assessment based on simultaneously acquired imaging and functional information corresponding to a target ROI.

### DRAWINGS

These and other features, aspects, and advantages of the present disclosure will become better understood when the following detailed description is read with reference to the accompanying drawings in which like characters represent like parts throughout the drawings, wherein:

FIG. 1 is a schematic representation of an exemplary imaging system that allows for simultaneous measurement of diagnostic parameters and interventional imaging, in accordance with aspects of the present disclosure;

FIG. 2 is a schematic representation of an exemplary embodiment of an integrated interventional device for use in simultaneous measurement of diagnostic parameters and interventional imaging, in accordance with aspects of the present disclosure;

FIG. 3 is a graphical representation of exemplary phantoms depicting changes in flow velocity due to different percentage area of stenosis, in accordance with aspects of the present disclosure; and

FIG. 4 is a flow diagram illustrating an exemplary method for performing an interventional procedure, in accordance with aspects of the present disclosure.

### DETAILED DESCRIPTION

The following description presents systems and methods for combining measurement of diagnostic parameters with interventional imaging using an integrated interventional device. Particularly, certain embodiments illustrated herein describe methods and systems that use the integrated interventional device for simultaneously acquiring three-dimensional (3D) geometry and volumetric information corresponding to a region of interest (ROI) in a vascular structure of a subject. Simultaneous acquisition of 3D geometry and volumetric information allows for greater accuracy in determining structural and functional characteristics that may be used for clinical assessment of the vascular structure. Particularly, the simultaneously acquired information may be used to aid in interventional procedure planning, execution, and/or post-procedure evaluation.

Although the following description is discussed with reference to IVUS imaging, certain embodiments of the present methods and systems may also be implemented in connection with other types of catheter-based interventional imaging systems, such as Optical Coherence Tomography (OCT) systems, transesophageal echocardiography (TEE), and Intra-cardiac Echocardiography (ICE) systems. Particularly, the systems and methods described herein find use, for example, in improving detection of coronary artery lesion and other anomalies in heart, thyroid, liver, or other organs of the subject by providing more accurate structural and functional measurements. Additionally, the present systems and methods may allow for accurate diagnosis and staging of coronary artery disease and monitoring of therapies including high-intensity focused ultrasound (HIFU), radiofrequency ablation (RFA), and brachytherapy.

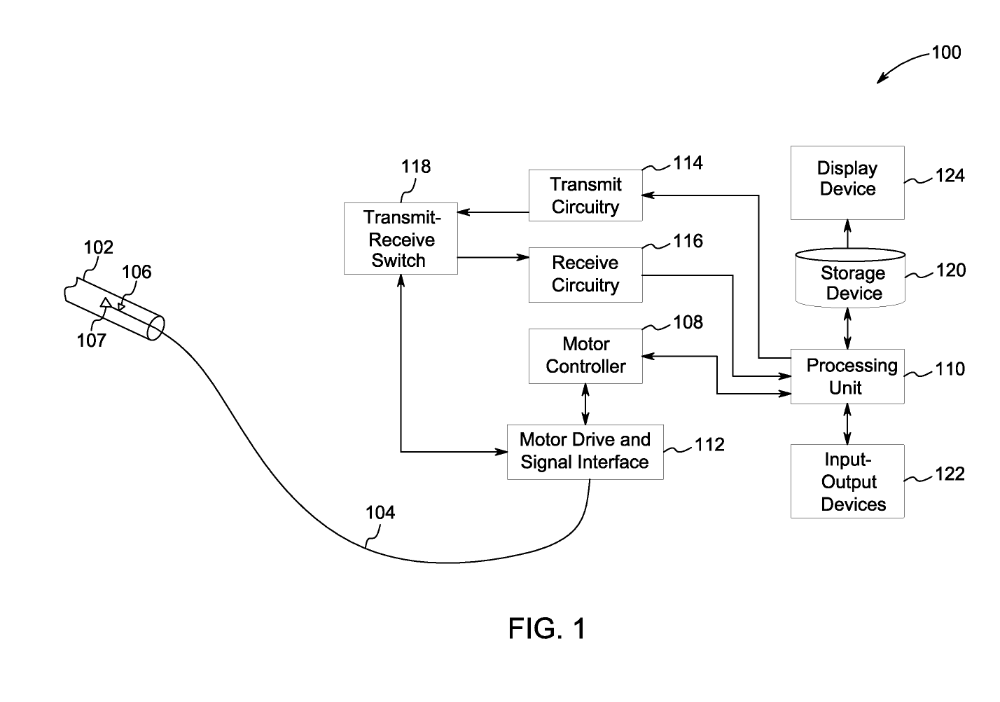

In certain embodiments, the present systems and methods may also be used for non-medical purposes, such as for nondestructive testing of fluid delivery systems, assessing leaks and blockages, and/or estimating differential pressure in pipes and other non-biological objects. An exemplary environment that is suitable for practicing various implementations of the present system is described in the following sections with reference to FIG. 1.

FIG. 1 illustrates an exemplary interventional imaging system 100. In one embodiment, the system 100 may be configured for imaging, providing a functional evaluation, and/or therapy to one or more target locations in biological tissues and/or non-biological regions of interest (ROIs). For discussion purposes, the system 100 is described with reference to an IVUS system for use in diagnosing and/or treating a patient. However, as previously noted, in certain embodiments, the system 100 may be implemented as certain other interventional imaging systems such as an OCT and/or ICE system. Additionally, it may be noted that although the present embodiment is described with reference to a blood vessel 102, certain embodiments of the system 100 may be used with other biological tissues of the patient such as lymph vessels, cerebral vessels, hepatic vessels, renal vessels, and/or other objects suitable for ultrasound imaging and flow measurement.

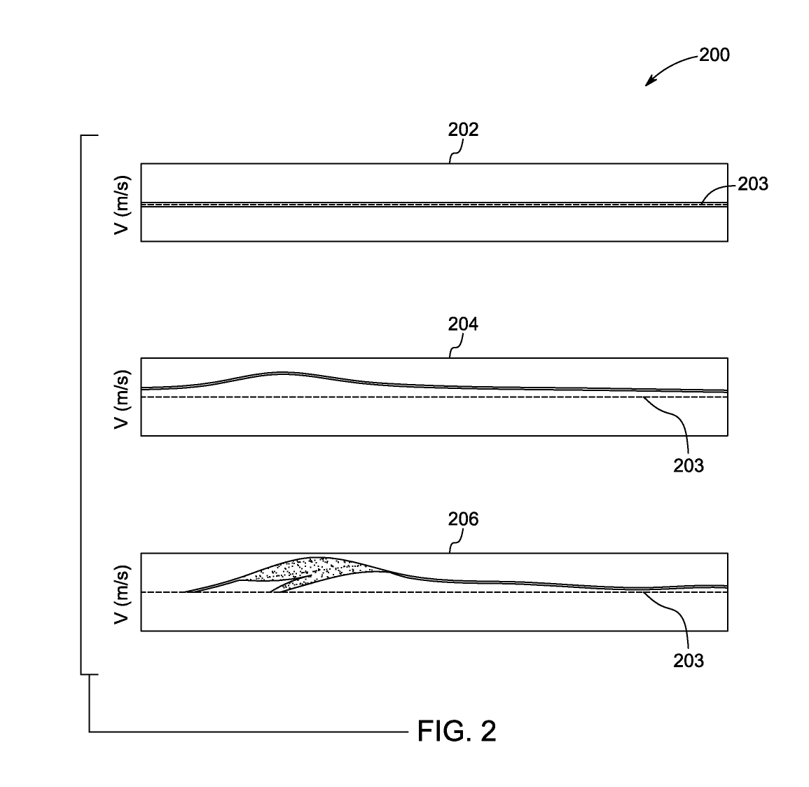

In one embodiment, the system 100 includes an integrated interventional device such as a catheter 104 adapted for use in a confined medical or surgical environment such as a body cavity, orifice, or the blood vessel 102. The catheter 104 may further include at least one image sensor 106 and at least one forward-looking flow measurement device or sensor 107 disposed at a distal end of the catheter 104. In one embodiment, the image sensor 106 acquires data for generating cross-sectional images of the blood vessel 102, whereas the flow sensor 107 measures one or more functional characteristics of the blood flowing through the blood vessel 102. Exemplary embodiments of the catheter 104, the image sensor 106, and the flow sensor 107 will be described in greater detail with reference to FIG. 2.

In one embodiment, the catheter 104, including the image sensor 106 and the flow sensor 107, is inserted into the target blood vessel 102 through one or more small incisions for reducing patient recovery time. In certain embodiments, the image sensor 106, for example, is configured to rotate inside the blood vessel 102 under control of a motor controller 108 and/or a processing subsystem 110 via a motor drive and signal interface 112. Additionally, the motor controller 108 and/or the processing subsystem 110 may also control operation of the forward-looking flow sensor 107 to generate ultrasound pulses of a desired frequency and/or repetition rate for use in estimating blood flow characteristics. Particularly, the motor controller 108 and/or the processing subsystem 110 may provide control and timing signals for controlling one or more operating parameters for either or both of the image sensor 106 and the flow sensor 107. The operating parameters, for example, may include a delivery sequence of different sets of ultrasound pulses, frequency of delivering the pulses, a time delay between two different pulses, beam intensity, and/or other operational parameters corresponding to the system 100.

In certain embodiments, the system 100 further includes transmit circuitry 114 and receive circuitry 116 for transmitting and receiving ultrasound signals. Further, the transmit circuitry 114 and receive circuitry 116 may be electrically coupled to the image sensor 106 and/or the flow sensor 107 through a transmit-receive switch 118 and/or the motor drive and signal interface 108. In one embodiment, the transmit circuitry 114, under control of processing subsystem 110, generates a pulsed electrical signal having a peak-to-peak voltage (Vpp) of about 50-80 volts to drive the image sensor 106 and/or the flow sensor 107 to emit ultrasound pulses into a desired ROI (not shown). For example, the transmit circuitry 114 may be configured to generates a pulsed electrical signal having a peak-to-peak voltage (Vpp) of about 50-80 volts to drive the image sensor 106 and/or the flow sensor 107 to emit ultrasound pulses. Particularly, in one embodiment, the pulsed electrical signal may be generated in one or multiple cycles at the center frequency of transducer (40-80 MHz) corresponding to the image sensor 106. At least a portion of the ultrasound pulses is reflected from the ROI to produce reflected ultrasound pulses that returns to the image sensor 106 and the flow sensor 107. The flow sensor 107 may convert the reflected ultrasound pulses into electrical signals that may pass through the motor drive and signal interface 112 and/or the transmit-receive switch 118 to the receive circuitry 116 for further processing.

In one embodiment, the receive circuitry 116 communicates the received electrical signals to the processing subsystem 110 that processes the received signals according to a plurality of selectable ultrasound modalities in real-time and/or off-line mode. To that end, the processing subsystem 110 includes devices such as one or more application-specific processors, digital signal processors, microcomputers, microcontrollers, Application Specific Integrated Circuits (ASICs) and/or Field Programmable Gate Arrays (FPGAs). The processing subsystem 110 may also include devices in communication with other components of the system 100 such as a picture archiving and communications system (PACS), a radiology department information system, hospital information system and/or to an internal or external communications network (not shown).

In certain embodiments, the processing subsystem 110 stores the received signals and/or processed information along with the delivery sequence, repetition frequency, time delay, intensity, imaging system parameters, and/or other operational data in a storage device 120 for further processing. The storage device 120, for example, may include devices such as a random access memory, a read-only memory, a disc drive, a solid-state memory device, and/or a flash memory. In certain embodiments, the storage device 120 may also store commands and inputs received from an operator during the interventional procedure.

Accordingly, in one embodiment, the processing subsystem 110 may be coupled to one or more user input-output devices 122, such as a keyboard, touchscreen, microphone, mouse, buttons, switches, audio devices, and/or video devices to receive the operator input and commands. In an exemplary implementation, the processing subsystem 110 allows the operator to select one or more ROIs and/or imaging parameters, for example, using a graphical user interface on a local or remote display device 124. In one embodiment, the display device 124 is communicatively coupled to the processing subsystem 110 and/or the input output devices 122. Further, the imaging parameters, for example, may include a rate of rotation of the imaging sensor 106, a velocity or length of a pullback of the catheter 104, a selection of structural and/or functional parameters for display, and one or more desired properties of resulting images.

In one embodiment, the processing subsystem 110 communicates the operator inputs to one or more of the transmit circuitry 114 and the motor controller 108. Accordingly, the image sensor 106, rotating under the control of the motor controller 108, emits ultrasound pulses towards one or more desired portions of the region surrounding the image sensor 106 to allow generation of a plurality of imaging lines. These imaging lines may be used collectively to reconstruct a two-dimensional (2D) and/or a three-dimensional (3D) cross-sectional image of the desired ROI, such as the walls of the blood vessel 102 and the tissue surrounding the blood vessel 102.

In one example, the processing subsystem 110 displays the 2D and/or 3D images along with corresponding patient data on the display device 124 for use in review, diagnosis, analysis, and/or treatment of the patient. In another example, the processing subsystem 110 stores the 2D and/or 3D images for later review and analysis, and/or communicates these images to another location for further evaluation.

In certain embodiments, a medical practitioner may employ the 2D and/or 3D images for determining structural characteristics corresponding to the ROI in the target blood vessel 102. For example, the 2D and/or 3D images may be used to identify tissue morphology including locations of a stenosis, lesions, vessel tree, and/or percentage narrowing at different points within the lumen of the blood vessel 102. The structural characteristics, in turn, may be used for detecting a pathological condition such as presence of plaque or other blockages, and/or for aiding in deploying a vascular stent.

However, as previously noted, the structural characteristics derived from the generated images may not be sufficient for identifying ischemia-causing lesions and/or for making certain other procedure-related decisions. Accordingly, in one embodiment, the processing subsystem 110 may be configured to provide control and timing signals to the forward-looking flow sensor 107 for acquisition of hemodynamically significant data corresponding to the ROI. Particularly, the processing subsystem 110 may provide control and timing signals to the forward-looking flow sensor 107 for generating a repetitive sequence of pulses that may be transmitted along the same scan line for generating M-mode data corresponding to blood flow through the target blood vessel 102.

In one embodiment, the processing subsystem 110 estimates functional characteristics for the target ROI based on the received signals. For example, when estimating intensity M-mode data, the processing subsystem 110 may use each received signal along a particular scan line for determining intensities. For color M-mode data, the processing subsystem 110 may evaluate a group of transmitted and received signals along the scan line for estimating flow characteristics such as velocity, volume, and/or pressure.

Moreover, in certain embodiments, the flow sensor 107 is configured to acquire the functional data simultaneously with the image data at a designated time, for example, at the same instant of time. However, in other embodiments, the designated time may correspond to different instants during a designated period of time, such as within 1-2 seconds, selected based on user input and/or control signals received from the processing subsystem 110. Accordingly, as used herein, the terms “simultaneously” “designated time,” and any variations thereof, may be used to refer to the same instant of time, and/or the same designated period of time.

Furthermore, the processing subsystem 110 may evaluate a combination of the structural information acquired using the image sensor 106 with volumetric information acquired using the flow sensor 107 to provide a more informed assessment of the target ROI. For example, the flow information and vessel dimensions estimated from the Doppler flow sensor and the IVUS images respectively, may be modeled to determine clinically relevant parameters such as percent diameter stenosis and/or percent area stenosis in the blood vessel 102. Generally, the percent area stenosis is estimated by comparing an area of a lesion or narrowing to a reference area in a normal segment of the blood vessel 102, which is proximal the lesion, as a percentage reduction. Typically, the percent area stenosis and/or the percent diameter stenosis are indicative of a severity of stenosis or occlusion of the vessel and are conventionally measured using X-ray imaging.

Embodiments of the present disclosure, however, allow for calculation of the percent area stenosis without use of additional X-ray imaging. Particularly, the processing subsystem 100 may be configured to calculate the percent area stenosis based on a ratio of the velocity values measured by the integrated catheter 104 in real-time. More specifically, the processing subsystem 110 may be configured to calculate the percent area stenosis based on a ratio of measured blood velocity in a normal segment (normal blood velocity) to blood velocity in a stenotic segment of the blood vessel 102 (intrastenotic blood velocity).

To that end, in one embodiment, the blood volume in a normal segment of the blood vessel, Qₙₒᵣₘₐₗ may be computed using equation (1):

Qₙₒᵣₘₐₗ=Vₙₒᵣₘₐₗ×Areaₙₒᵣₘₐₗ (1)

where Vₙₒᵣₘₐₗ corresponds to the blood velocity measured in the normal segment of the blood vessel 102 and Areaₙₒᵣₘₐₗ corresponds to the luminal area corresponding to the normal segment of the blood vessel 102 determined using IVUS images.

Similarly, the blood volume in a stenotic segment of the blood vessel, Qₛₜₑₙₒₛᵢₛ, may be computed using equation (2):

Qₛₜₑₙₒₛᵢₛ=Vₛₜₑₙₒₛᵢₛ×Areaₛₜₑₙₒₛᵢₛ (2)

where Vₛₜₑₙₒₛᵢₛ corresponds to the blood velocity measured in the stenotic segment of the blood vessel 102 using the Doppler flow sensor 107 and Areaₛₜₑₙₒₛᵢₛ corresponds to the luminal area corresponding to the stenotic segment of the blood vessel 102.

Typically, a decrease in area corresponding to the stenosis may result in an increase in stenotic velocity Vₛₜₑₙₒₛᵢₛ. FIG. 2, for example, illustrates a graphical representation 200 depicting exemplary phantoms indicative of changes in flow velocity due to different percentage of stenosis. For example, in FIG. 2, reference numeral 202 corresponds to a phantom that shows steady flow of fluid through a 50-millimeter segment of a vessel in the absence of stenosis. The steady fluid flow in the phantom 202 may be selected as reference flow 203. Further, reference numeral 204 corresponds to another phantom that depicts a sharp increase in the velocity with respect to the reference flow 203 following a stenosis that causes 30% occlusion in the blood vessel. Moreover, reference numeral 206 corresponds to yet another phantom that depicts a sharper increase in the velocity and turbulence following a stenosis that causes 50% occlusion in the blood vessel.

With returning reference to FIG. 1, although the stenosis causes an increase in the blood velocity, since a volume of blood flowing through the blood vessel 102 remains the same before and after the stenotic portion, the normal blood volume Qₙₒᵣₘₐₗ is substantially equal to intrastenotic blood volume Qₛₜₑₙₒₛᵢₛ. Accordingly, the ratio of normal and intrastenotic blood velocities may be equated to the ratio of area of the stenosis to the area of the normal segment, as defined by equation (3):

$$\frac{{V}_{normal}}{{V}_{stenosis}} = \frac{{Area}_{stenosis}}{{Area}_{normal}} \qquad ( 3 )$$

Further, the percent area stenosis may be determined, for example, using equation (4):

$$Percent Area Stenosis = 100 \times ( 1 - \frac{{V}_{normal}}{{V}_{stenosis}} ) \qquad ( 4 )$$

Additionally, as area has a square root relationship with diameter, the percent narrowing of a diameter of the blood vessel 102 may be computed, for example, using equation (5):

$$Percent Diameter Stenosis = 100 \times ( 1 - \sqrt{\frac{{V}_{normal}}{{V}_{stenosis}}} ) \qquad ( 5 )$$

As previously noted, the percent diameter stenosis provides a clinically useful indication of the extent of occlusion of the blood vessel 102, in turn, allowing for prescription of a suitable treatment for the patient.

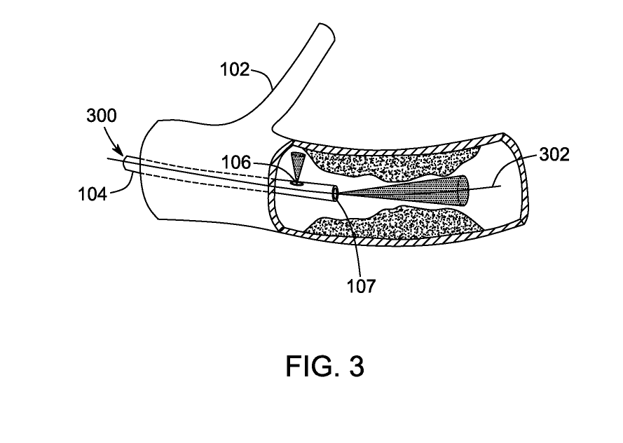

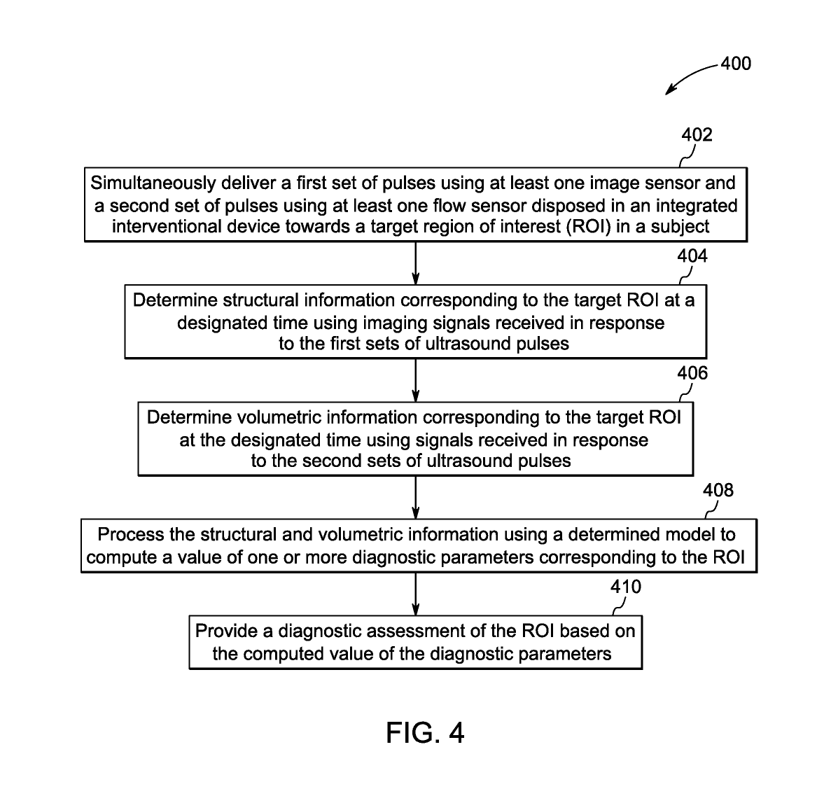

Alternatively, in certain embodiments, the processing subsystem 110 may be configured to process the structural and volumetric information using certain other determined models to estimate clinically significant diagnostic parameters, as discussed in detail with reference to FIGS. 3-4. The determined model, in one embodiment, may correspond to a CFD model. Accordingly, in one embodiment, the processing subsystem 110 may be configured to construct a CFD model to compute the FFR values in real-time. Particularly, the processing subsystem 110 may be configured to determine structural information such as 2D and/or 3D geometry corresponding to the target blood vessel from the IVUS images and provide the determined geometry as input to the CFD model. The geometry, for example, may include inlet diameter, length, and diameter of the vascular structure, location, stiffness, and elasticity of the ROI, and/or diameter, length, input angle, extent, and/or composition of a lesion. Further, the processing subsystem 110 may also be configured to input ultrasound flow profile to the CFD model to provide flow boundary conditions for computing a transstenotic pressure across one or more detected lesions. In one embodiment, the flow profile may include, for example, velocity, volume, and/or pressure information. Additionally, in certain embodiments, imaging, perfusion, and/or pressure information acquired using other imaging modalities such as MRI, CT, and/or a pressure sensing device may be input to the CFD model to allow estimation of upstream and downstream pressure in the blood vessel 102 with respect to the lesion.

Particularly, in one embodiment, the CFD model employs the structural and volumetric information along with specified boundary conditions to determine pressure values throughout a defined domain, such as along a designated length of a blood vessel. For example, the processing subsystem 100 may be configured to use the pressure values determined at positions before (proximal) and/or after (distal) a stenotic lesion to compute the FFR or pressure gradient across the lesion. In scenarios, where a lesion is present in a secondary branch of the blood vessel, the processing subsystem 110 may use the CFD model to estimate the pressure gradient at the extents of each segment leading up to the secondary branch. The processing subsystem 100 may then communicate the FFR value computed by the CFD model to a medical practitioner audibly or visually on the display device 124 for use in providing a diagnosis of the ROI of the subject.

Although FIG. 1 illustrates a specific number of exemplary components, in certain embodiments, the system 100 may include fewer or additional components for use in interventional imaging, data processing, and/or for allowing automation of the interventional procedure. In one example, the system 100 may include additional devices such as one or more analog-to-digital-converters (ADC), filters, amplifiers and/or switching subsystems for use in data processing. Alternatively, one or more of the components such as the motor controller 108, the processing subsystem 110, the transmit circuitry 114, and/or the receive circuitry 116 may be combined into a single or fewer devices, thus optimizing floor space in the interventional procedure room.

Embodiments of the present system 100, thus, provide the interventional practitioner and/or the automated system 100 with greater functionality and efficiency to perform the interventional procedure. Particularly, simultaneous measurement of structural and functional characteristics using the integrated catheter 104 including both the image sensor 106 and the flow sensor 107 simplifies the workflow of the interventional procedure. The simplified workflow, in turn, aids in reducing the procedure time and improving data accuracy and patient comfort. Certain examples of the structure and functioning of the integrated catheter 104 for use in efficient interventional procedures will be described in greater detail with reference to FIG. 2.

FIG. 3 illustrates an exemplary embodiment of an integrated interventional device 300 for use in simultaneous interventional imaging and diagnostic measurements. In certain embodiments, the device 300 includes a catheter, such as the catheter 104 of FIG. 1. In one embodiment, the catheter 104, for example, may be a mechanical or a phased-array catheter including a manual and/or a mechanized pullback mechanism (not shown). For example, the catheter 104 may have a length of about 1-1.5 meters and may include a rotational shaft having a diameter of about 0.6 millimeter (mm) diameter disposed inside a plastic tube having a diameter of about 1.2 mm. Furthermore, as previously noted with reference to FIG. 1, the catheter 104 may include at least one image sensor 106 and the at least one flow sensor 107 disposed at a distal end of the catheter 104 for allowing for simultaneous interventional imaging and diagnostic measurements. In one embodiment, the image sensor 106 corresponds to an IVUS sensor having a size of about 0.5 mm² and a thickness of about 20-100 micrometers, whereas the flow sensor 107 corresponds to a Doppler sensor having a similar size and thickness as the image sensor 106. In an alternative embodiment, however, the image sensor 106 and the flow sensor 107 may have different sizes and thicknesses.

In certain embodiments, the catheter 104 may be operatively coupled to a guide wire 302 for aiding in insertion of the catheter 104 into an access site that is proximal or distal from the intravascular ROI (target ROI). In one example, the guide wire 302 is a thin wire with a flexible tip that may be guided through the blood vessel 102 to a region that is proximal to the blockage or stenosis. In certain embodiments, the interventional practitioner controls the movement and direction of the guide wire 302 by gently manipulating the end of the guide wire 302 that is present outside the patient's body. Particularly, in one embodiment, the interventional practitioner manipulates the guide wire so as to pass the catheter 104 over the guide wire 302 and position the distal end of the catheter 104 close to the lesion, blockage, or stenosis 204.

The insertion and movement of the catheter 104 within the blood vessel 102, however, is a challenging procedure. Accordingly, in certain embodiments, the integrated device 300 may be communicatively coupled to an interventional imaging system and/or a tracking system (not shown in FIG. 2) for providing guidance for navigating the catheter 104 and/or the guide wire 302 through the blood vessel 102 without causing injury to the surrounding tissues. Additionally, when traversing a tortuous vessel, the distal end of the catheter 104 may be positioned proximal the target ROI, such as close to a site of the stenosis 204. Once positioned, the image sensor 106 and/or the flow sensor 107 in the catheter 104 may be configured to transmit and receive ultrasound signals along one or more scan lines for simultaneously imaging surrounding tissues and determining blood flow characteristics, respectively.

In certain embodiments, the image sensor 106 may be a side-looking ultrasound transducer configured to generate a 2D and/or 3D cross-sectional image of the stenotic portion 204 of the blood vessel 102. Particularly, in one embodiment, the image sensor 106 may include a single-element miniaturized transducer configured to rotate mechanically about the catheter 104, offering side-looking capabilities. Additionally, the size of the transducer, for example, may be in the range of 0.25 to 1.0 mm. Moreover, the transducer may be flat or curved, disc-shaped, block-shaped, spherical, or ring-shaped based on specific imaging requirements. In certain embodiments, the shape and size of the transducer may be selected such that the image sensor 106 is suitable to be inserted, placed, and/or advanced inside the patient's body without significant tissue disruption.

Although FIG. 2 depicts the image sensor 106 as including a single-element transducer, in an alternative embodiment, the image sensor 106 may include a multi-element array of transducers such that the resultant imaging can provide 2D and/or a 3D radial cross-sectional image of the vessel wall. In yet another embodiment, instead of an ultrasound transducer, the image sensor 106 may include an optical device suitable for optical imaging. Furthermore, in certain embodiments, the image sensor 106 may be configured to produce a forward-looking image and/or a side-looking image of the imaged vessel. To that end, the integrated device 300 may include electric and/or mechanical steering means to allow multiple degrees of freedom corresponding to a distal tip of the catheter 104. Particularly, using the steering means, the image sensor 106 may transmit and receive ultrasound signals along one or more scan lines for acquiring image data corresponding to a 360-degree view of the target ROI.

Similarly, in certain embodiments, the flow sensor 107 may also use the steering means to acquire, for example, color M-mode data corresponding to a region proximal a stenosis 204. To that end, in one embodiment, the flow sensor 107 may be a forward-looking ultrasound Doppler sensor configured to acquire the M-mode data to determine one or more characteristics of the blood flow, such as blood volume and velocity, while traversing the stenosis 204. In another embodiment, the flow sensor 107 may include certain other flow measurement devices such as pressure sensors, temperature sensors, flow nozzles, venture tubes, and/or orifice plates.

Further, in one embodiment, an associated processing system, such as the processing subsystem 110 of FIG. 1, may use the determined velocity information to compute a pressure gradient or FFR value across the stenosis in the blood vessel 102. Generally, FFR measurements account for a contribution of collateral vessels and provide a threshold of cutoff values for discriminating between ischemic and hemorrhagic lesions. As previously noted, in one embodiment, an FFR value of less than 0.75 may be considered to signify presence of an ischemic lesion.

Conventionally, CFR and FFR are measured during catheterization using separate CFR and FFR catheters, each including a guide wire with a pressure-sensing transducer that is passed across a coronary lesion. Subsequent to induction of maximal hyperemia using a vasodilation agent such as intravenous or intra-arterial adenosine, the pressure gradient across the coronary lesion is recorded and FFR is calculated as a mean distal coronary pressure divided by mean aortic pressure. However, due to the invasive nature of the procedure, the need for pharmacologic vasodilation, and risks related to instrumentation of the blood vessels, conventional FFR measurements find limited use in clinical environments.

In contrast, the device 300 allows for accurate measurement of the pressure gradient across the stenosis 204 without the use of a separate CFR or FFR catheters that adds to patient discomfort and procedure time. Particularly, the integrated interventional device 300 allows for simultaneous acquisition of imaging and volumetric information to provide the interventional practitioner with accurate real-time structural and/or volumetric information for planning, executing, and/or evaluating the interventional procedure with greater accuracy. Certain exemplary methods for such simultaneous measurement of diagnostic parameters and intravascular imaging using the integrated interventional device are discussed in greater detail with reference to FIG. 4.

FIG. 4 illustrates a flow chart 400 depicting an exemplary method for performing an interventional procedure. The exemplary method may be described in a general context of computer executable instructions stored and/or executed on a computing system or a processor. Generally, computer executable instructions may include routines, programs, objects, components, data structures, procedures, modules, functions, and the like that perform particular functions or implement particular abstract data types. The exemplary method may also be practiced in a distributed computing environment where optimization functions are performed by remote processing devices that are linked through a wired and/or wireless communication network. In the distributed computing environment, the computer executable instructions may be located in both local and remote computer storage media, including memory storage devices.

Further, in FIG. 4, the exemplary method is illustrated as a collection of blocks in a logical flow chart, which represents operations that may be implemented in hardware, software, or combinations thereof. The various operations are depicted in the blocks to illustrate the functions that are performed, for example, during the steps of determining structural and volumetric information, computing diagnostic parameters using a determined model, and providing a diagnostic assessment of the ROI in the exemplary method. In the context of software, the blocks represent computer instructions that, when executed by one or more processing subsystems, perform the recited operations.

The order in which the exemplary method is described is not intended to be construed as a limitation, and any number of the described blocks may be combined in any order to implement the exemplary method disclosed herein, or an equivalent alternative method. Additionally, certain blocks may be deleted from the exemplary method or augmented by additional blocks with added functionality without departing from the spirit and scope of the subject matter described herein. For discussion purposes, the exemplary method will be described with reference to the elements of FIGS. 1-2.

Generally, investigation of a stenosis entails both structural and functional assessment of an affected region. Typically, the structural assessment may be performed using angiography, IVUS imaging, and/or optical imaging. The measured structural characteristics may be used, for example, to compute percent occlusion of the blood vessel 102 of FIG. 1. The computed occlusion values may then be employed as an indication of stenosis severity. However, as previously noted, a more accurate determination of the nature and extent of the stenosis entails use of volumetric information including pressure and flow characteristics prevalent at the site of the stenosis.

Certain conventional interventional systems have been known to employ FFR estimates to ascertain if the detected stenosis warrants stenting. However, these conventional systems employ separate IVUS and FFR catheters for measuring structural and functional parameters, respectively. Use of such conventional systems, therefore, entails multiple insertions and retractions of separate IVUS and FFR catheters, which may result in deviation from a desired measurement site in the vessel, thus causing measurement errors. Additionally, the multiple measurements using the different catheters may also lead to longer procedure time, use of pharmacological vasodilation, greater patient discomfort, injury, and/or equipment costs.

Accordingly, embodiments of the present method describe a technique for performing an interventional procedure using a single integrated interventional device that allows for simultaneous imaging and measurement of diagnostic parameters. For discussion purposes, an exemplary embodiment of the present method will be described with reference to an interventional procedure for ascertaining a need for revascularization at a target ROI such as a site of stenosis in a blood vessel by determining corresponding structural and volumetric information.

At step 402, a first set of pulses are simultaneously delivered using at least one image sensor with a second set of pulses delivered using at least one flow sensor in an integrated interventional device towards a target ROI in a subject. For discussion purposes, the present embodiment is described with reference to use of IVUS for imaging the target ROI. However, in alternative embodiments, other types of imaging modalities may also be used to image the target ROI. For example, in one embodiment, OCT may be employed for imaging the target ROI using a plurality of optical pulses.

In an exemplary implementation, the integrated interventional device corresponds to the IVUS catheter 104 of FIG. 1 or the device 300 of FIG. 3. Further, the integrated IVUS device may include at least one image sensor, such as the image sensor 106 of FIG. 1 and at least one forward-looking flow sensor such as the flow sensor 107 of FIG. 1 disposed at the distal end of the catheter 104. In certain embodiments, the image sensor may also include or incorporate one or more volumetric measurement devices such as pressure sensors, flow sensors, and/or temperature sensors for determining volumetric information corresponding to the target ROI.

As previously noted, the image sensor 106 and the flow sensor 107 may be configured to deliver the first and second set of ultrasound pulses simultaneously at the same instant of time or during the same designated period of time. To that end, the timing and frequency of delivery of the first and second set of signals by the image sensor and/or the flow sensor may be controlled by a processing system, such as the processing subsystem 110 of FIG. 1. In one embodiment, the processing subsystem may provide the timing and control signals based on a designated protocol and/or user specified inputs.

Further, at step 404, structural information corresponding to the ROI at a designated instant or period of time is determined using signals received in response to the first sets of pulses. In one embodiment, the structural information may be derived from the one or more images corresponding to the target ROI that may be reconstructed using the imaging signals received in response to the first set of pulses delivered by the image sensor. To that end, the image sensor may provide side-looking and/or forward-looking imaging capabilities. Specifically, the image sensor may be configured to generate high-frequency sound waves that reflect off tissue or vessel walls and may be used to create 2D and/or 3D cross-sectional images from within the vessel for visualizing structural information corresponding to the imaged vessel in offline and/or real-time mode.

In accordance with exemplary aspects of the present disclosure, the structural information may include features of interest in the vascular structure that may necessitate further evaluation. For example, the structural information may include anatomical characteristics such as stiffness, elasticity, inlet diameter, length and diameter of the blood vessel, and/or location, diameter, length, input angle, extent and/or composition of a lesion. The structural information may further include location of a vessel tree and/or a percent area stenosis of the vessel, as determined at the designated time. Collectively, the structural information may define a 3D geometry of the target ROI that may be further correlated with corresponding volumetric information.

Accordingly, at step 406, the volumetric information corresponding to the target ROI may be determined at the designated time based on reflected signals received in response to the second set of pulses. As previously noted, the volumetric information including flow characteristics may be determined using the flow sensor. In one embodiment, the flow sensor may be a forward-looking ultrasound Doppler sensor configured to determine volumetric information within the blood vessel. In certain embodiments, the Doppler flow sensor may be configured to process the received signals to derive a spatial representation that describes one or more flow characteristics of blood through the desired blood vessel.

Particularly, in one embodiment, the Doppler flow sensor may be used to generate spectral or color-flow type Doppler information corresponding to one or more aspects of blood flow or velocity within the target ROI being imaged. In an alternative embodiment, however, the flow sensor may include a pressure sensor configured to directly measure a pressure gradient across the stenosis. In certain embodiments, the processing system configures the flow sensor to transmit the second set of signals having a desired wavelength and/or frequency at designated instants of time. The echo signals received in response to the second set of pulses may be processed by the processing subsystem to determine flow characteristics such as a velocity profile of the flow of blood through the blood vessel of interest in a time and/or a space domain.

To that end, in one embodiment, the processing subsystem may be configured to perform a first-stage digital filtering of the received signals for removing noise and/or slow moving objects from the received signals. The digital filtering, for example, of the received signals may be performed using a band-pass or a wall filter. Further, the processing subsystem may calculate a power spectrum in a direction of transmission of the second set of ultrasound pulses and/or obtain a phase shift difference in the direction of transmission. Additionally, the processing subsystem may also perform a second-stage digital filtering for removing noise from the filtered signal. The frequency and/or the phase shift information, thus calculated, may be used to determine velocity of the blood flow at a particular location in the target ROI.

In an alternative embodiment, however, the velocity profile may be determined using the reconstructed IVUS images. Particularly, the processing subsystem may determine the velocity profile from the ultrasound signals received in response to the first set of ultrasound pulses delivered by the image sensor. To that end, the processing subsystem may be configured to perform a first-stage digital filtering of the received imaging signals for removing noise and/or slow moving objects. Further, the processing subsystem may calculate a correlation length between successive ultrasound pulse transmissions. Subsequently, the velocity may be calculated by dividing the width of an ultrasound beam in the first set of ultrasound pulses by an average of correlation length and an interval between the successive ultrasound transmissions.

Further, at step 408, the structural and volumetric information is processed using a determined model to compute a value of one or more diagnostic parameters corresponding to the ROI. As used herein, the term “diagnostic parameters” refers to parameters such as a pressure gradient, FFR, or CFR that provide an indication of a pathological condition of the target ROI. Additionally, the values of the diagnostic parameters may be representative of one or more functional characteristics of the ROI before, during, and/or subsequent to an interventional procedure, thereby providing an indication of an efficacy of an implant or therapy.

For example, in one embodiment, estimation of a pressure gradient or FFR across a site of stenosis in the blood vessel may be determined to aid in a revascularization decision. Accordingly, embodiments of the present disclosure employ a determined model to process the structural information along with an estimated velocity flow profile in the ROI to compute transstenotic pressure gradient or PPR. Particularly, in one embodiment, the pressure gradient may be determined using an empirical model. In one example, the empirical model may be defined using equation (6).

$$Δ p = ρ ( \frac{\partial u}{\partial t} + u \frac{\partial u}{\partial x} ) \qquad ( 6 )$$

where Δp corresponds to the pressure gradient, ρ corresponds to fluid density, u corresponds to velocity, t corresponds to time, and x corresponds to distance along the vessel of interest.

As depicted in equation (6), the pressure gradient computation entails use of a derivative of velocity, for example, in a temporal and/or a spatial domain. Additionally, the measured velocity may be multiplied by a derivative of the measured velocity as a function of distance along a scan line. The resulting product, in turn, may be summed with the temporal derivative of the velocity. This summed value may then be multiplied with the density of the fluid flowing through the vessel to estimate the pressure gradient. Thus, equation (6) may be used to compute the pressure gradient for each spatial location from corresponding velocity values.

It may be noted that equation (6) provides one illustrative example for computing the pressure gradient. In another example, a the pressure gradient may be estimated using another empirical model such as a Young and Tsai model that uses simple inputs such as an area percentage of stenosis, length of lesion, blood velocity entering stenosis, volume flow rate, and/or inlet and exit angles to calculate the distal end pressure. An exemplary computation of the FFR using a particular Young and Tsai model may be represented using equation (7).

$$\frac{Δ p}{ρ {U}^{2}} = \frac{{K}_{v}}{{R}_{e}} + \frac{{K}_{t}}{2} {( \frac{{K}_{v}}{{R}_{e}} - 1 )}^{2} \qquad ( 7 )$$

where Δp corresponds to a pressure drop across a selected length of a vascular structure, ρ corresponds to fluid density, Kᵥ, and Kₜ correspond to experimentally determined coefficients dependent on stenotic geometry determined from the data acquired by the image sensor, and Rₑ corresponds to Reynold's number.

Typically, the empirical models, such as defined using equations (6) and (7), may be configured to estimate the distal pressure assuming steady and laminar fluid flow to allow for shorter computation times. However, oversimplifying actual flow conditions may preclude accurate reproduction of physical effects prevailing at the location of the lesion, thus providing only approximate pressure measurements.

Accordingly, in certain implementations that entail accurate measurements, computational fluid dynamics (CFD) modeling may be used to compute the pressure gradient and/or other diagnostic parameters. In one embodiment, CFD modeling entails constructing an accurate patient-specific anatomical model of a vessel of interest such as a coronary artery and a physical model indicative of physical phenomena, such as motion, laminar flow, turbulence, and/or enthalpy, associated with the blood flow during hyperemia. The CFD modeling further entails defining a numerical solution that allows computation of the pressure gradient or FFR corresponding to a feature of interest such as a lesion in the vessel based on laws of physics governing fluid dynamics.

In one embodiment, constructing the anatomical model entails extracting structural information corresponding to the diseased vessel from the reconstructed ultrasound images using image segmentation algorithms. The image segmentation algorithms extract structural characteristics of the blood vessel, for example, by determining a topology of a corresponding vessel tree, identifying, analyzing and segmenting lesions or plaques in the blood vessel, and/or delineating the structural boundary.

Further, construction of the physical model may entail generating a finite element mesh having a plurality of vertices and elements from the anatomical model. Moreover, the flow information determined from the Doppler ultrasound data may be divided into discrete elements corresponding to the mesh. Additionally, boundary conditions that specify fluid behavior and fluid interaction properties at boundaries of the imaging problem may also be defined.

In certain embodiments, the anatomical and physical models, thus constructed, may be coupled with the fluid dynamics-based numerical solution to provide a realistic model of fluid flow. For example, realistic modeling of coronary flow requires coupling of an anatomical model of the heart, systemic circulation, and coronary circulation to a patient-specific model of the aortic root and epicardial coronary arteries that is extracted from the ultrasound images. The different models, for example, combine peripheral resistance, blood vessel compliance, and other suitable factors into distinct elements dependent on specific parameters of each circulatory bed. The combination of factors may then be used in the numerical solution to provide an estimation of the pressure gradient or FFR values at different points in the blood vessel of the patient.

Particularly, in one embodiment, a set of governing equations founded in a relationship between conservation of mass and momentum balance in fluid dynamics may be used to compute the FFR value using the numerical solution. The set of governing equations, for example, correspond to the “Navier Stokes equations” that may be solved for the FFR value as a function of position (three spatial coordinates) and time. Generally, the governing equations correspond to nonlinear partial differential equations that are solved analytically under highly idealized circumstances. However, for a realistic representation of patient-specific diseased vessels, CFD modeling may provide the numerical solution that approximates the governing equations to solve for the pressure values at a finite but large number of points. In particular, the numerical solution may entail computation of millions of nonlinear equations simultaneously and repeating this process for thousands of time intervals in a single cardiac cycle.

Furthermore, in addition to the use of the governing equations, the boundary conditions that interface the anatomical model to the remainder of the circulation may also be defined to construct the patient-specific computational blood flow. In certain embodiments, the boundary conditions may correspond to mathematical relationships between one or more variables of interest, such as blood flow and pressure gradient, defined on determined boundaries of the CFD model. For example, when modeling blood flow in coronary arteries, the boundary conditions may correspond to lateral arterial surface, inlet boundary of the aortic root, and/or outlet boundaries of the ascending aorta and the coronary arteries.

Thus, the anatomical model, the physical model, and the boundary conditions, in combination may be used to solve the governing equations corresponding to blood flow for determining pressure values at different points along the vessel. Particularly, in one embodiment, the governing equations may be solved by normalizing a mean hyperemic pressure field by an average mean hyperemic pressure in the aorta. The resulting solution may provide a spatial distribution of FFR along a desired length of the blood vessel.

Conventionally, FFR is known to be independent of hemodynamic conditions, such as heart rate, blood pressure, or contractility, and thus provides a useful indication of a physiological severity of a stenosis in a blood vessel. As previously noted, FFR values of less than 0.75 indicate presence of a functionally significant stenosis that would necessitate stenting or revascularization of the affected blood vessel. In contrast, FFR values of greater than 0.80 indicate absence of ischemia, thereby favoring a prescription of medical therapy over revascularization. Accordingly, the computed FFR value may be used for further diagnosis of the patient.

Particularly, at step 410, a diagnostic assessment of the ROI may be provided based on the computed value of the diagnostic parameters. The diagnostic assessment may allow a better understanding of a pathological condition of the target ROI and facilitate decisions regarding prescription of subsequent therapeutic procedures. For example, the pressure gradient, along with the measured structural characteristics may be used as a useful indication of stenosis severity, thereby aiding in determining a need for revascularization of the blood vessel. Similarly, other functional parameters may be used to assess a health condition of the subject, such as a structural or functional anomaly in the tissues indicative of a pathological condition. In certain embodiments, additional confirmatory and/or complementary data may be used, for example, within a Bayesian decision-making framework to improve a positive predictive value and/or negative predictive value of the diagnostic assessment.

Embodiments of the present systems and methods, thus, describe use of an integrated IVUS catheter for use in simultaneous imaging and functional assessment of a subject. Particularly, use of a single catheter improves a probability of measuring the imaging and functional data at the same time and location in the vessel of interest, thus allowing for a more accurate correlation between the structural and functional information. Further, combining the real-time structural and functional information using a CFD model allows for a more accurate flow and volume estimation using a single interventional device, while precluding a need for additional imaging or medication.

In particular, integrating a forward-looking Doppler sensor into the IVUS catheter allows for flow volume and velocity measurements without having to advance the catheter across the stenosis and impeding the flow of blood through the blood vessel as experienced when using a conventional FFR catheter. The flow measurements obtained using a single integrated interventional device, in turn, may be used along with the structural information to estimate a plurality of diagnostic parameters such as FFR, CFR, percent area stenosis, and pressure gradients, thus obviating need to measure pressure values on a distal side of a stenosis to estimate a pressure gradient across the stenosis. Accurate computation of the pressure gradients, in turn, may allow accurate identification of a nature of a lesion and determination of a functional significance of the identified lesion via a single interventional procedure.

Moreover, the forward-looking ultrasound flow sensor may also provide information corresponding to a flow channel ahead of the flow sensor to provide guidance for catheterization. Furthermore, use of the integrated catheter obviates a need for separate IVUS and FFR catheters, each of which typically costs hundreds of dollars, thus optimizing equipment and/or operational costs. Additionally, use of the single integrated catheter precludes multiple insertions and retractions, thereby simplifying interventional workflow, reducing the procedure time, and/or enhancing patient comfort as compared to conventional interventional systems.

It may be noted that although specific features of various embodiments of the present systems and methods may be shown in and/or described with respect to only certain drawings and not in others, this is for convenience only. It is to be understood that the described features, structures, and/or characteristics may be combined and/or used interchangeably in any suitable manner in the various embodiments, for example, to construct additional assemblies and techniques. Furthermore, the foregoing examples, demonstrations, and process steps, for example, those that may be performed by the motor controller 108 and the processing subsystem 110 may be implemented by a single device or a plurality of devices using suitable code on a processor-based system.

It should also be noted that different implementations of the present disclosure may perform some or all of the steps described herein in different orders or substantially concurrently, that is, in parallel. In addition, the functions may be implemented in a variety of programming languages, including but not limited to Python, C++, or Java. Such code may be stored or adapted for storage on one or more tangible, machine-readable media, such as on data repository chips, local or remote hard disks, optical disks (that is, CDs or DVDs), solid-state drives, or other media, which may be accessed by a processor-based system to execute the stored code.

While only certain features of the present disclosure have been illustrated and described herein, many modifications and changes will occur to those skilled in the art. It is, therefore, to be understood that the appended claims are intended to cover all such modifications and changes as fall within the true spirit of the present disclosure.

1. An interventional imaging system, comprising:
an integrated interventional device, comprising:
at least one image sensor adapted for use in target region of a subject and configured to produce a first set of pulses for determining structural information corresponding to the target region at a designated time;
at least one flow sensor configured to produce a second set of pulses for use in determining volumetric information corresponding to the target region at the designated time, wherein one or more of the image sensor and the flow sensor are positioned at the distal end of the integrated interventional device;
at least one processing subsystem operatively coupled to the integrated interventional device and configured to:
process the structural and volumetric information using a determined model to compute one or more diagnostic parameters corresponding to the target region; and
provide a diagnostic assessment of the target region based on the computed diagnostic parameters.

2. The system of claim 1, wherein the integrated interventional device comprises an intravascular ultrasound catheter.

3. The system of claim 1, wherein the image sensor comprises a side-looking ultrasound sensor.

4. The system of claim 1, wherein the image sensor comprises an optical device.

5. The system of claim 1, wherein the integrated interventional device comprises a pressure sensor, a flow sensor, and a temperature sensor, or combinations thereof, integrated into the image sensor.

6. The system of claim 1, wherein the flow sensor comprises a forward-looking Doppler sensor.

7. The system of claim 1, further comprising a display device configured to display the structural information, the volumetric information, the diagnostic parameters, the diagnostic assessment, or combinations thereof.

8. A method for performing an interventional procedure, comprising:
simultaneously delivering a first set of pulses using at least one image sensor and a second set of pulses using at least one flow sensor, the image sensor and the flow sensor being disposed in an integrated interventional device, wherein the first set of pulses and the second set of pulses are directed towards a target region in a subject;
determining structural information corresponding to the target region at a designated time using imaging signals received in response to the first set of pulses;
determining volumetric information corresponding to the target region at the designated time using flow signals received in response to the second set of pulses;
processing the structural and volumetric information using a determined model to compute one or more diagnostic parameters corresponding to the target region; and
providing a diagnostic assessment of the target region based on the computed diagnostic parameters.

9. The method of claim 8, wherein the first and second set of pulses comprises one or more ultrasound pulses.

10. The method of claim 8, wherein the first and second set of pulses comprise one or more optical pulses.

11. The method of claim 8, wherein determining the structural information comprises determining three-dimensional geometry of the target region.

12. The method of claim 8, wherein determining the structural information comprises determining at least one of an inlet diameter of a vascular structure in the target region, a length of the vascular structure, a diameter of the vascular structure, a location of the vascular structure, a stiffness of the target region, an elasticity of the target region, a diameter of a blockage in the vascular structure, a length of the blockage, an input angle corresponding to the blockage, a composition of the blockage, or combinations thereof.

13. The method of claim 8, wherein determining the volumetric information comprises calculating a velocity profile of a fluid flowing through the target region in one or more of a time domain and a space domain.

14. The method of claim 13, wherein calculating the velocity profile comprises:
digitally filtering the flow signals received in response to the second set of pulses delivered by the flow sensor;
computing one or more of a power spectrum and a phase shift difference in a direction of transmission of the second set of pulses; and
determining a velocity of the fluid flowing through the target region based on the computed power spectrum, the phase shift information, or a combination thereof.

15. The method of claim 8, wherein the determined model comprises an empirical model or a computational fluid dynamics model corresponding to the target region.

16. The method of claim 8, wherein computing one or more diagnostic parameters comprises determining a fractional flow reserve value, a pressure gradient, a blood flow volume, a coronary flow reserve value, or combinations thereof, corresponding to the target region.

17. The method of claim 8, wherein providing the diagnostic assessment of the target region comprises displaying, the structural information, the volumetric information, and the diagnostic parameters, one or more images corresponding to the target region, or combinations thereof, on a display device.

18. The method of claim 8, wherein providing the diagnostic assessment of the target region comprises providing the structural information, the volumetric information, diagnostic parameters, or combinations thereof, audibly through an audio device.

19. The method of claim 8, wherein providing a diagnostic assessment of the target region comprises computing a percent area stenosis, a percent diameter stenosis, or a combination thereof, based on the volumetric information determined from the second set of pulses.

20. A non-transitory computer readable medium that stores instructions executable by one or more processors to perform an interventional procedure, comprising:
simultaneously delivering a first set of pulses using at least one image sensor and a second set of pulses using at least one flow sensor, the image sensor and the flow sensor being disposed in an integrated interventional device, wherein the first set of pulses and the second set of pulses are directed towards a target region in a subject;
determining structural information corresponding to the target region at a designated time using imaging signals received in response to the first set of pulses;
determining volumetric information corresponding to the target region at the designated time using flow signals received in response to the second set of pulses;
processing the structural and volumetric information using a determined model to compute one or more diagnostic parameters corresponding to the target region; and
providing a diagnostic assessment of the target region based on the computed diagnostic parameters.
